(19) United States
(12) Patent Application Publication
(10) Pub. No.: US 2024/0302242 A1
(43) Pub. Date: Sep. 12, 2024

(54) COLORIMETRIC SENSOR FOR DETECTING HARMFUL GAS LEAKAGE IN PRODUCT MANUFACTURING PROCESS AND METHOD FOR MANUFACTURING COLORIMETRIC SENSOR

(71) Applicant: IDKLAB INC., Daejeon (KR)

(72) Inventors: Sung-Ho SHIN, Daejeon (KR); Won Tae HWANG, Daejeon (KR); Hwan Soo KIM, Daejeon (KR)

(21) Appl. No.: 18/289,260

(22) PCT Filed: Apr. 19, 2022
(86) PCT No.: PCT/KR2022/005591

§ 371 Date: Nov. 2, 2023

(30) Foreign Application Priority Data
May 4, 2021 (KR) 10-2021-0057905
Apr. 7, 2022 (KR) 10-2022-0043314

(51) Int. Cl. G01M 3/20

(52) U.S. Cl. CPC G01M 3/20

(57) ABSTRACT

Disclosed are a colorimetric sensor for detecting harmful gas leakage in a product manufacturing process and a method for manufacturing the colorimetric sensor. The colorimetric sensor according to an embodiment may comprise: a colorimetric composition formed such that a dye including a metal component of which the own color changes when reacting with a harmful gas is bound to submicron fibers in the form of fine particles; and an adhesive band of a mesh structure to which the colorimetric composition is bound.

### TECHNICAL FIELD

Embodiments of the present disclosure relate to a colorimetric sensor for detecting the leakage of a harmful gas in a product manufacturing process and a method of manufacturing the colorimetric sensor.

### BACKGROUND ART

Safety accidents attributable to gas leakage in a piping and processing facility that is used in a product manufacturing process, such as a semiconductor process or a display process, frequently occur. A person regularly performs environment safety and patrols in order to detect and early monitor a harmful gas, but it is difficult to accurately check a gas leakage portion through only a portable instrument or a fixed sensor because a gas is invisible to the eye.

For example, most of semiconductor type gas sensors use a change in the electrical conductivity, which occurs when a gas comes into contact with a surface of a ceramic semiconductor. Metal oxide (ceramics) that is stable at a high temperature is basically used in the semiconductor type gas sensor because most of semiconductor type gas sensors are heated and used in the atmosphere. Metal oxide basically has the properties of a semiconductor. If metal atoms are excessive (oxygen deficiency), a semiconductor becomes an n type semiconductor. If the metal atoms are deficient, a semiconductor becomes a p type semiconductor.

However, such a semiconductor type gas sensor has problems in that it cannot detect the leakage of a harmful gas in real time because a person has to regularly perform patrols and check possible leakage points of piping and processing facilities while carrying the gas sensor, in the case of a portable instrument, and a fixed sensor is high in price and has a low degree of determination.

### DISCLOSURE

### Technical Problem

There are provided a colorimetric sensor capable of detecting the leakage of a harmful gas in a product manufacturing process, such as a semiconductor process or a display process, and a method of manufacturing the colorimetric sensor.

### Technical Solution

There is provided a colorimetric sensor including a colorimetric composition in which a dye including a metal component having a unique color changed upon reaction with a harmful gas has been bound to submicron fibers in a fine particle form, and an adhesive band having a mesh structure to which the colorimetric composition has been bound.

According to an aspect, the submicron fibers may have a first fiber having a first thickness and a second fiber having a thickness relatively thicker than the first thickness intersected and formed, whereby a space for a spread and adsorption of a gas may be formed between the first fibers by the second fiber.

According to another aspect, at least some of the submicron fibers may be aligned and formed in one direction in order to increase the density of the fibers.

According to still another aspect, the harmful gas may include at least one of Cl₂, BCl₃, BF₃, HBr, SiH₄, NH₃, HCl steam, HF steam and TMAH steam, the metal component may include at least one of Cu, Co, Ni, Fe, Zn, Mn, and Pb as a form of an ion or salt, and the dye further may include at least one of an acetate group, a chlorine group, and a nitrate group

According to still another aspect, the adhesive band having a mesh structure is a double-sided adhesive band, and may have one surface bound to the colorimetric composition and the other surface bound to a piping and processing facility for a product manufacturing process.

According to still another aspect, a colorimetric accelerator may be further bound to the submicron fibers.

According to still another aspect, the colorimetric accelerator may include at least one of particulates, polymeric beads, ceramic particles, and ionic liquids that provide surface texturing.

There is provided a method of manufacturing a colorimetric sensor, including steps of manufacturing a colorimetric composition including submicron fibers to which a dye including a metal component having a unique color changed upon reaction with a harmful gas has been bound, and binding the colorimetric composition to an adhesive band having a mesh structure.

According to an aspect, the step of manufacturing the colorimetric composition may include steps of manufacturing an electrospinning solution including a dye, polymers, and a solvent, and manufacturing a submicron fiber membrane to which the dye has been bound in a fine particle form by electrospinning the electrospinning solution.

According to another aspect, the step of manufacturing the colorimetric composition may include steps of manufacturing an electrospinning solution including polymers, and a solvent, manufacturing a submicron fiber membrane by electrospinning the electrospinning solution, and coating the dye on a fiber including the submicron fiber membrane.

According to still another aspect, the step of coating the dye may include coating the dye by dropping a solution in which the dye has been molten to a fiber including the submicron fiber membrane in a vacuum filtration manner.

### Advantageous Effects

The colorimetric sensor capable of detecting the leakage of a harmful gas in a product manufacturing process, such as a semiconductor process or a display process, and the method of manufacturing the colorimetric sensor can be provided.

### DESCRIPTION OF DRAWINGS

The accompany drawings, which are included as part of the detailed description in order to help understanding of the present disclosure, provide embodiments of the present disclosure and describe the technical characteristics of the present disclosure along with the detailed description.

FIG. 1 is a flowchart illustrating an example of a method of manufacturing a colorimetric sensor according to an embodiment of the present disclosure.

FIG. 2 is a diagram illustrating an example of a process of manufacturing a colorimetric sensor according to an embodiment of the present disclosure.

FIG. 3 is a diagram illustrating an example of a process of detecting the leakage of a harmful gas through the colorimetric sensor in an embodiment of the present disclosure.

FIGS. 4 to 7 are images illustrating examples of colorimetric sensors that have been manufactured according to embodiments of the present disclosure.

FIG. 8 is an image illustrating an example in which the colorimetric sensor manufactured according to an embodiment of the present disclosure is used.

FIG. 9 shows images illustrating an example of a change in the color of the colorimetric sensor manufactured according to an embodiment of the present disclosure.

FIG. 10 shows images illustrating another example of a change in the color of the colorimetric sensor manufactured according to an embodiment of the present disclosure.

FIG. 11 shows images illustrating another example of a change in the color of the colorimetric sensor manufactured according to an embodiment of the present disclosure.

FIG. 12 shows images illustrating an example of a change in the color of a PVA submicron fiber colorimetric sensor to which a DPD dye was bound in an embodiment of the present disclosure.

FIG. 13 shows images illustrating another example of a change in the color of a PVA submicron fiber colorimetric sensor to which a DPD dye was bound in an embodiment of the present disclosure.

FIG. 14 shows images illustrating an example of a change in the color of the colorimetric sensor using an FeCl₂ dye in an embodiment of the present disclosure.

FIG. 15 shows images illustrating examples of a color change process in a colorimetric sensor using a DPD dye, a colorimetric sensor using copper acetate, and a colorimetric sensor using FeCl₂ in an embodiment of the present disclosure.

FIGS. 16 to 18 are images illustrating examples of a color change attributable to the influence of moisture in an embodiment of the present disclosure.

FIG. 19 is a diagram illustrating an example of a color change process of a submicron fiber membrane by a combination with a harmful gas in an embodiment of the present disclosure.

FIG. 20 is an image illustrating an example of a submicron fiber membrane on which a dye was uniformly coated in an embodiment of the present disclosure.

FIG. 21 shows images illustrating an example of a submicron fiber membrane to which a dye was bound in an embodiment of the present disclosure.

FIG. 22 is a diagram illustrating an example in which a colorimetric accelerator was used in an embodiment of the present disclosure.

### BEST MODE

The present disclosure may be modified in various ways and may have various embodiments. Specific embodiments are hereinafter described in detail with reference to the accompanying drawings.

In describing the present disclosure, a detailed description of the known functions and constructions will be omitted if it is deemed to make the subject matter of the present disclosure unnecessarily vague.

Terms, such as a “first” and a “second”, may be used to describe various components, but the components are not restricted by the terms. The terms are used to only distinguish one component from the other components.

Hereinafter, a colorimetric sensor capable of detecting the leakage of a harmful gas in a product manufacturing process, such as a semiconductor process or a display process, and a method of manufacturing the colorimetric sensor are described in detail with reference to the accompanying drawings.

In an embodiment of the present disclosure, a colorimetric composition capable of checking the leakage of a toxic process gas (e.g., Cl₂, BCl₃, BF₃, HBr, SiH₄, NH₃, HCl steam, HF steam, or TMAH steam) that is used in a product manufacturing process, such as a semiconductor process or a display process, at a high speed may be provided. Such a colorimetric composition may include a metal-acetate-series dye, and can detect the leakage of a harmful gas in a way that a unique color of a metal component is changed because a new metal compound is formed through a reaction between the metal component included in the dye and a gas component upon reaction with the target gas. In this case, a three-dimensional colorimetric composition that facilitates the spread of the gas therein and that may cause a change in the three-dimensional stereoscopic color through a combination of an electrically spinned submicron fiber and a color change dye can be provided.

The provided colorimetric composition may be bound to an adhesive band having a mesh structure. In this case, in order to bind the colorimetric composition to a piping and processing facility for a product manufacturing process, the colorimetric composition may be bound to the adhesive band having the mesh structure that has a relatively wider area than the colorimetric composition. In this case, the colorimetric sensor including the colorimetric composition and the adhesive band may be used to detect the leakage of a gas by being applied to a piping or process facility for a product manufacturing process, such as a fitting part, a pipe connection part, a gauge connection part, around a gasket, a bushing seal, a drive rod, and a welding part.

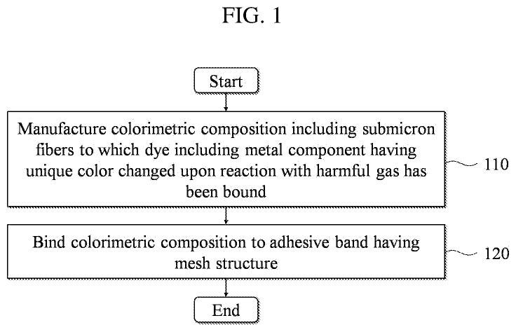

FIG. 1 is a flowchart illustrating an example of a method of manufacturing a colorimetric sensor according to an embodiment of the present disclosure. The method of manufacturing the colorimetric sensor according to the present embodiment may include step 110 of manufacturing a colorimetric composition including submicron fibers to which a dye including a metal component having a unique color changed upon reaction with a harmful gas has been bound, and step 120 of binding the colorimetric composition to an adhesive band having a mesh structure.

In step 110, as an embodiment, a colorimetric composition may be manufactured in a way to manufacture a submicron fiber membrane by using an electrospinning solution including a dye. For example, in step 110, after the electrospinning solution including a dye including a metal component having a unique color is changed upon reaction with a harmful gas, polymers, and a solvent is manufactured, the submicron fiber membrane to which the dye has been bound in the form of fine particles by electrospinning the electrospinning (E-spinning) solution may be manufactured as a colorimetric composition.

As another embodiment, in step 110, the colorimetric composition may be manufactured in a way to coat the dye on a fiber of the submicron fiber membrane manufactured by using the electrospinning solution. For example, in step 110, the electrospinning solution including polymers and a solvent may be manufactured. The submicron fiber membrane may be manufactured by electrospinning the electrospinning solution. In this case, the colorimetric composition may be manufactured by coating the dye on the fiber included in the submicron fiber membrane. A method of coating a solution in which the dye has been dissolved in ethanol in a vacuum filtration manner on submicron fibers by dropping the solution on a fiber included in the submicron fiber membrane may be used as the coating of the dye, but the present disclosure is not limited thereto.

In this case, the harmful gas may include at least one of Cl₂, BCl₃, BF₃, HBr, SiH₄, NH₃, HCl steam, HF steam, and TMAH steam. The metal component may include at least one of Cu, Co, Ni, Fe, MN, and Pb as a form of an ion or salt. Furthermore, the dye may further include at least one of an acetate group, a chlorine group, and a nitrate group.

In the embodiment of the electrical spinning, the submicron fibers may be manufactured so that a first fiber having a first thickness and a second fiber having a relatively greater thickness than the first thickness are intersected and formed. In this case, a space is formed between the first fibers each having a relatively smaller thickness than the second fiber having a relatively greater thickness, which may help the spread and adsorption of a gas. A technology for adjusting thickness of the fiber that is discharged through electrical spinning has already been known, and a detailed description thereof is omitted.

Furthermore, at least some of the submicron fibers may be aligned and formed in one direction in order to increase the density of the fibers. A technology for aligning fibers discharged through electrical spinning in one direction has already been known, and a detailed description thereof is omitted.

In step 120, as already described, the colorimetric composition and the adhesive band having a mesh structure may be bound. In this case, in order to bind the colorimetric composition to a piping and processing facility for a product manufacturing process, the colorimetric composition may be bound to the adhesive band having a mesh structure, which has a relatively wider area than the colorimetric composition.

According to an embodiment, a colorimetric accelerator may be further bound to the submicron fibers. The colorimetric accelerator may include at least one of particulates, polymeric beads, ceramic particles, and ionic liquids which provide surface texturing.

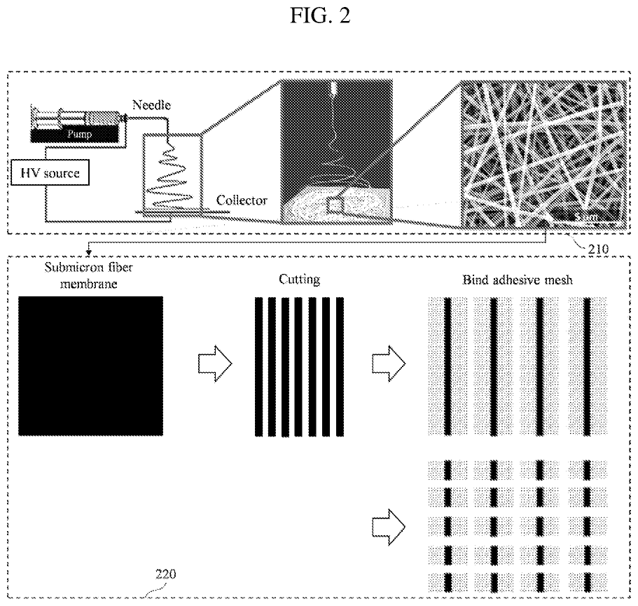

FIG. 2 is a diagram illustrating an example of a process of manufacturing a colorimetric sensor according to an embodiment of the present disclosure. In FIG. 2, a first dotted box 210 illustrates an example of an electrical spinning process. A second dotted box 220 illustrates an example in which the submicron fiber membrane manufactured through the electrical spinning process is bound to the adhesive band having a mesh structure. The second dotted box 220 illustrates an example in which the submicron fiber membrane is cut, then bound to the adhesive band, and cut in a proper size again. However, an embodiment in which the submicron fiber membrane is bound to the adhesive band having a large area and cut in a proper size at once may also be considered.

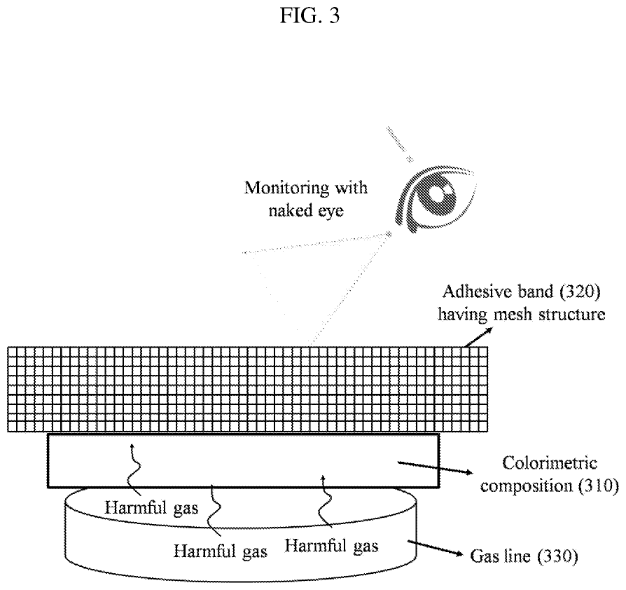

FIG. 3 is a diagram illustrating an example of a process of detecting the leakage of a harmful gas through the colorimetric sensor in an embodiment of the present disclosure. FIG. 3 illustrates an example in which a colorimetric composition 310 has been bound to a gas line 330 through an adhesive band 320 having a mesh structure. In this case, when a harmful gas leaks from the gas line 330, a unique color of a metal component may be changed because the leaking harmful gas reacts to the metal component included in the colorimetric composition 310. As the unique color of the metal component is changed, the color of the colorimetric composition 310 including the metal component may be changed. The leakage of the harmful gas may be detected because such a color change is monitored with the naked eye of a person.

In the embodiment of FIG. 3, it has been described that a person detects the leakage of a harmful gas by detecting a change in the color of the colorimetric composition 310 with the naked eye. However, the leakage of the harmful gas may be detected by automatically detecting a change in the color of the colorimetric composition 310 through CCTV or an addition colorimetric sensor.

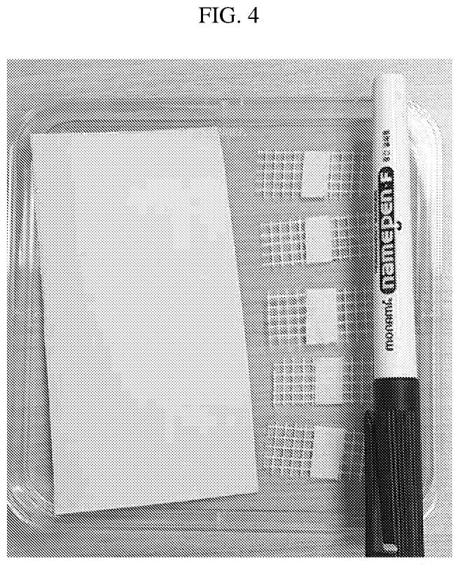

FIGS. 4 to 7 are images illustrating examples of colorimetric sensors that have been manufactured according to embodiments of the present disclosure.

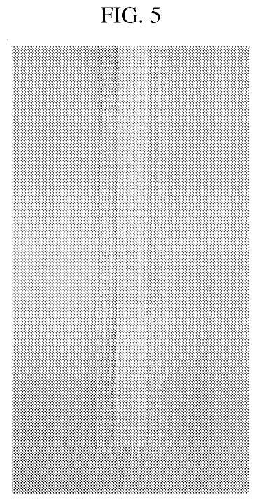

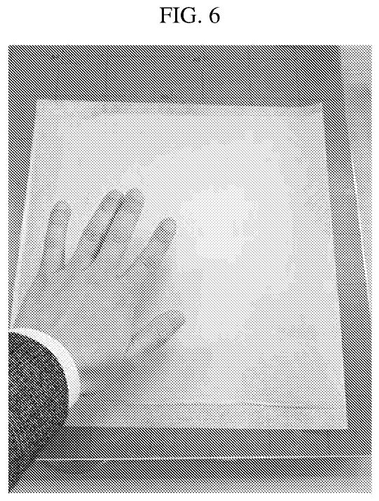

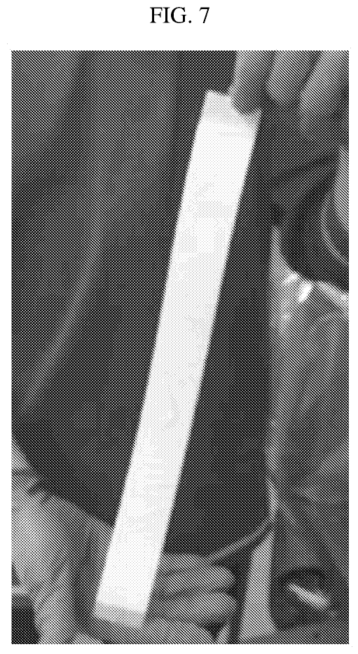

The image in FIG. 4 illustrates an example in which an adhesive band having a mesh structure was bound to a colorimetric composition and a colorimetric composition cut after the colorimetric composition was cut. Furthermore, the image in FIG. 5 illustrates another example in which an adhesive band having a mesh structure was bound to a colorimetric composition. Furthermore, the image in FIG. 6 illustrates an example in which a large-sized adhesive band having a mesh structure was bound to a colorimetric composition. The image in FIG. 7 illustrates an example in which the colorimetric composition to which the large-sized adhesive band having a mesh structure was bound in FIG. 6 was cut in a predetermined size.

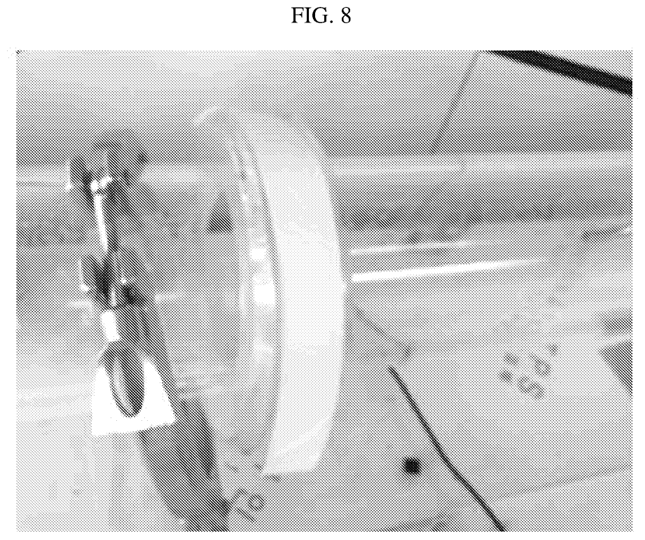

FIG. 8 is an image illustrating an example in which the colorimetric sensor manufactured according to an embodiment of the present disclosure is used. In this case, the image in FIG. 8 illustrates an example in which a colorimetric composition was attached to a connection port of a pipe through an adhesive band having a mesh structure. When a harmful gas leaks from the connection port of the pipe, a change in the color of the colorimetric composition may appear. Such a change in the color can be easily checked with the naked eye of a person through the adhesive band having a mesh structure.

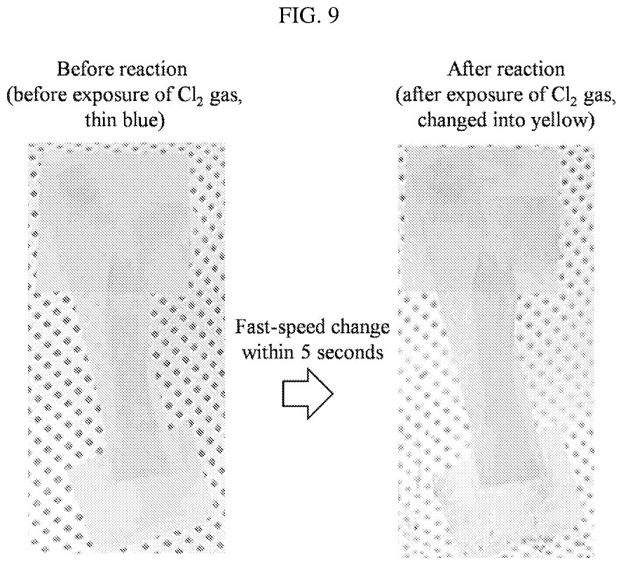

FIG. 9 shows images illustrating an example of a change in the color of the colorimetric sensor manufactured according to an embodiment of the present disclosure. The embodiment of FIG. 9 shows images obtained by monitoring a change in the color of a submicron fiber membrane before and after exposure when a Cl₂ gas was exposed to the submicron fiber membrane that was manufactured by exposing submicron fibers to which copper acetate was bound through electrical spinning 2 hours. In this case, it could be monitored that the color of the submicron fiber membrane of the present embodiment was changed from thin blue to yellow within 5 seconds.

In order to manufacturing the colorimetric sensor using copper acetate, first, the submicron fiber membrane may be manufactured by electrically spinning (e.g., a voltage 10 kV, a distance 15 cm, a needle gauge 25 G, a discharge rate 20 μl/min, and a discharge time 2 h to 4 h) a polymer nanofiber solution manufactured by mixing copper acetate hydrate 0.5 g and polyacrylonitrile (PAN) 0.35 g into an organic solvent 3.5 ml as a color dye.

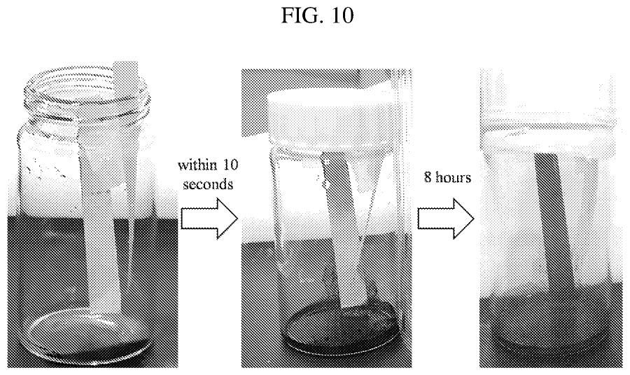

FIG. 10 shows images illustrating another example of a change in the color of the colorimetric sensor manufactured according to an embodiment of the present disclosure. The embodiment of FIG. 10 shows images obtained by monitoring a change in the color of the submicron fiber membrane according to exposure times when a Cl₂ gas was exposed to a submicron fiber membrane manufactured by electrically spinning submicron fibers to which copper acetate was bound for 4 hours. Even in the embodiment, it could be seen that the color of the submicron fiber membrane was changed even in short exposure within 10 seconds and a change in the color becomes more evident as the exposure time is increased.

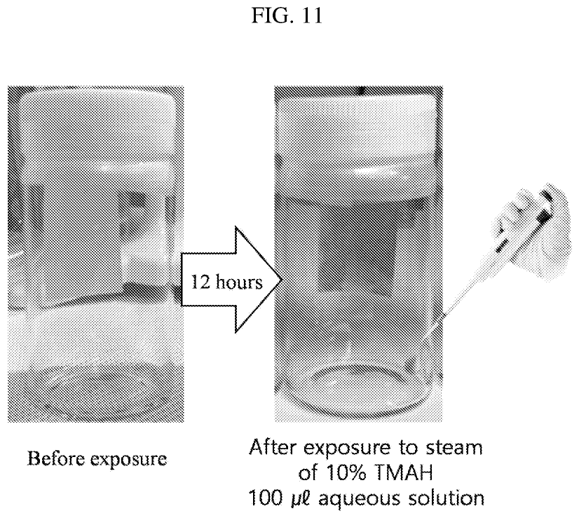

FIG. 11 shows images illustrating another example of a change in the color of the colorimetric sensor manufactured according to an embodiment of the present disclosure. The embodiment of FIG. 11 was obtained by monitoring a change in the color of a submicron fiber membrane before and after exposure when the steam of a 10% tetramethylammonium hydroxide (TMAH) aqueous solution is exposed to a submicron fiber membrane manufactured by using submicron fibers to which manganese acetate was bound. In the present embodiment, a color change was monitored after about 12 hours due to low volatility of the TMAH aqueous solution.

In order to manufacture the colorimetric sensor using manganese acetate, first, the submicron fiber membrane may be manufactured by electrically spinning (e.g., a voltage 8.5 kV, a distance 15 cm, a needle gauge 25 G, a discharge rate 30 μl/min, and a discharge time 4 h) a polymer nanofiber solution manufactured by mixing manganese acetate 0.5 g and polyacrylonitrile (PAN) 1 g into an organic solvent 8.5 ml as a color dye.

Hereinafter, an embodiment in which a polyvinyl alcohol (PVA) submicron fiber colorimetric sensor to which a diethyl-p-phenylendiamine (DPD) dye was bound is manufactured is described.

First, an electrospinning solution including DI water 18 g (=18 ml), PVA (polymerization degree) about 1500) 2 g, and DPD (N,N-Diethyl-p-phenylenediamine sulfate salt, >98%) 1.5 g was manufacture. The manufactured electrospinning solution was electrically spinned under conditions including a temperature of 22° C. (+2° C.), humidity of 23%(+2%), a needle gauge 25 G, a tip to collector distance (TCD) 6.5 cm, a voltage 9 kV, and a discharge rate 20 μl/min. In this case, electrically spinned submicron fibers were collected and the colorimetric composition was manufactured by using a nylon mesh and polyethylene terephthalate (PET) fabric as a substrate.

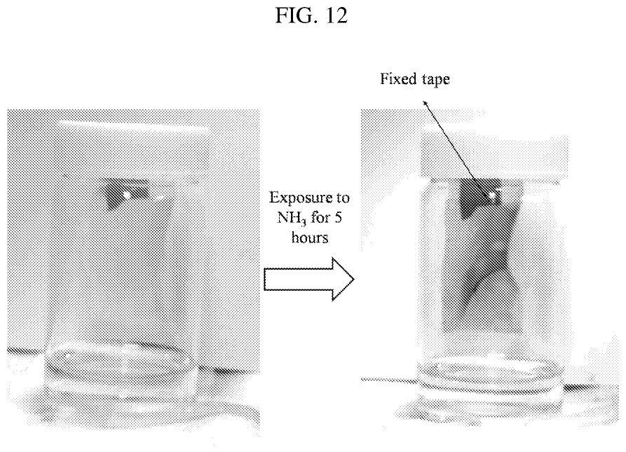

FIG. 12 shows images illustrating an example of a change in the color of a PVA submicron fiber colorimetric sensor to which a DPD dye was bound in an embodiment of the present disclosure. From the images in FIG. 11, it was checked that the color of the colorimetric sensor was changed into purple series after NH₃ was exposed to the PVA submicron fiber colorimetric sensor to which the DPD dye was bound. NH₃ was identified as alkalinity, and provides OH alkali through a reaction with water like “NH₃(g)+H₂O(l)→NH₃(aq)↔NH₄\hat{ }+(aq)+OH\hat{ }-(aq)”. It was determined that the colorimetric sensor showed an acid alkali reaction due to strong alkali because the colorimetric sensor was changed into purple due to NH₃.

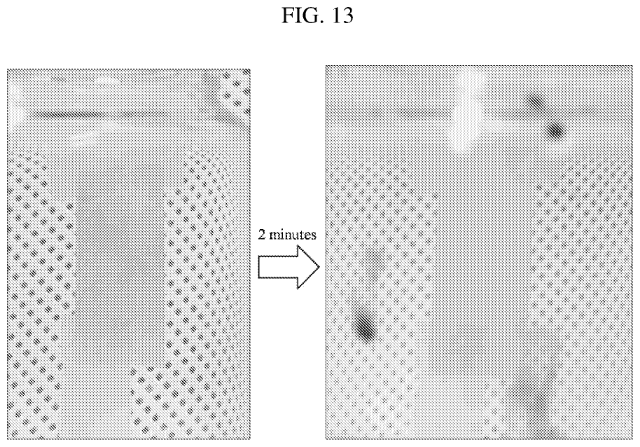

FIG. 13 shows images illustrating another example of a change in the color of a PVA submicron fiber colorimetric sensor to which a DPD dye was bound in an embodiment of the present disclosure. The images in FIG. 12 illustrate an example in which the colorimetric sensor was changed into yellow series after Cl₂ was exposed to the PVA submicron fiber colorimetric sensor to which the DPD dye was bound for 2 minutes.

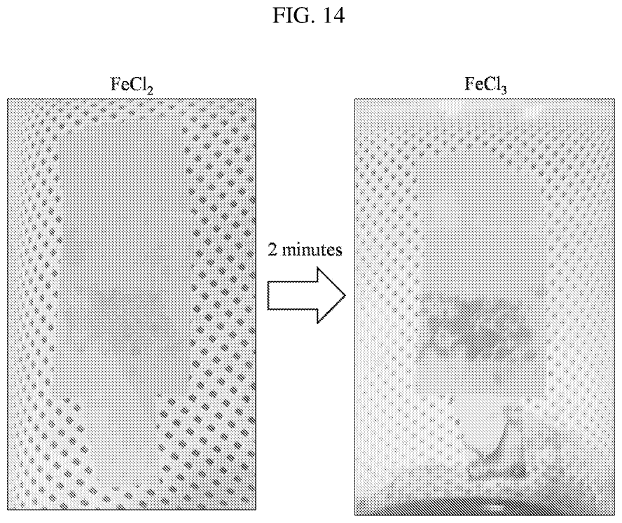

FIG. 14 shows images illustrating an example of a change in the color of the colorimetric sensor using an FeCl₂ dye in an embodiment of the present disclosure. The images in FIG. 14 illustrates an example in which the color of the colorimetric sensor was changed from green series to brown series after a Cl₂ gas was exposed to the colorimetric sensor using the FeCl₂ dye. The images show a difference between the color of commonly known FeCl₂ and the color of FeCl₃ and the color of commonly known FeCl₂ and the color of FeCl₃ are matched.

In order to manufacture the colorimetric sensor using the FeCl₂ dye, first, the submicron fiber membrane may be manufactured by electrically spinning (e.g., a voltage 10 kV, a distance 15 cm, a needle gauge 25 G, a discharge rate 20 μl/min, and a discharge time 2 h to 4 h) a polymer nanofiber solution manufactured by mixing polyacrylonitrile (PAN) 0.35 g into an organic solvent 3.5 ml. Thereafter, the colorimetric sensor using an FeCl₂ dye may be manufactured by preparing an FeCl₂ solution (e.g., mixing FeCl₂·4H₂O grains 0.35 g and ethanol 3.5 ml as a solvent) and dropping, into a PAN nano fiber layer, a solution in which FeCl₂ has been dissolved in ethanol in a vacuum filtration manner.

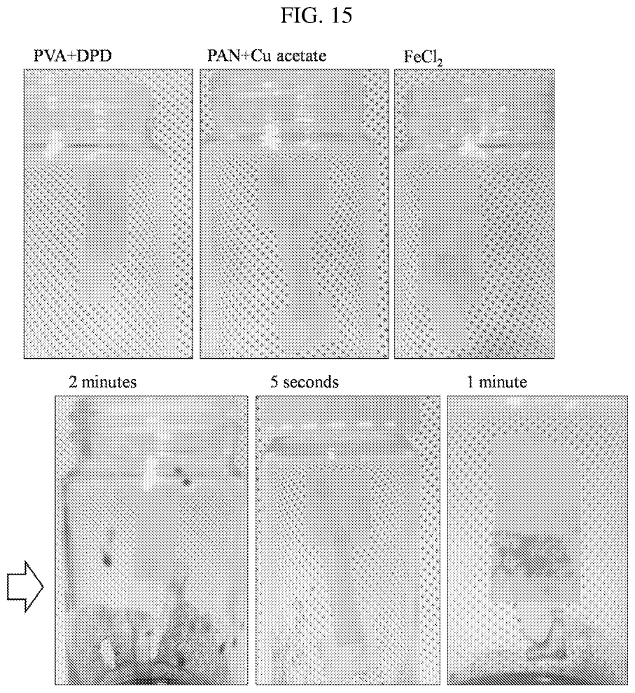

FIG. 15 shows images illustrating examples of a color change process in a colorimetric sensor using a DPD dye, a colorimetric sensor using copper acetate, and a colorimetric sensor using FeCl₂ in an embodiment of the present disclosure. The images in FIG. 15 illustrate examples of a color change when a Cl₂ gas was exposed to each of the colorimetric sensor using a DPD dye, the colorimetric sensor using copper acetate, and the colorimetric sensor using FeCl₂. The colorimetric sensor using copper acetate showed a color change with the fastest 5 second. Next, the colorimetric sensor using FeCl₂ had a colorimetric time of 1 minute, and the colorimetric sensor using the DPD dye had a colorimetric time of 2 minutes.

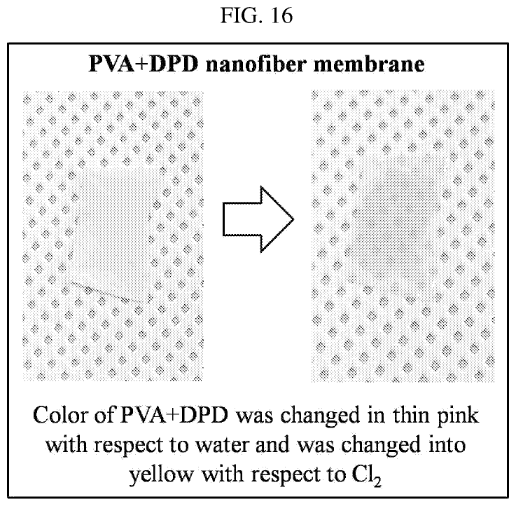

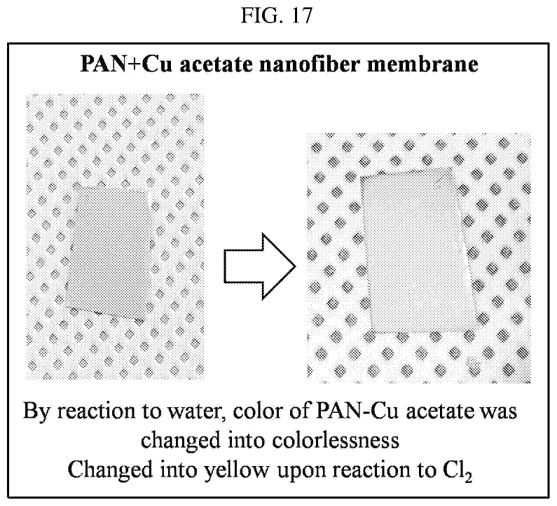

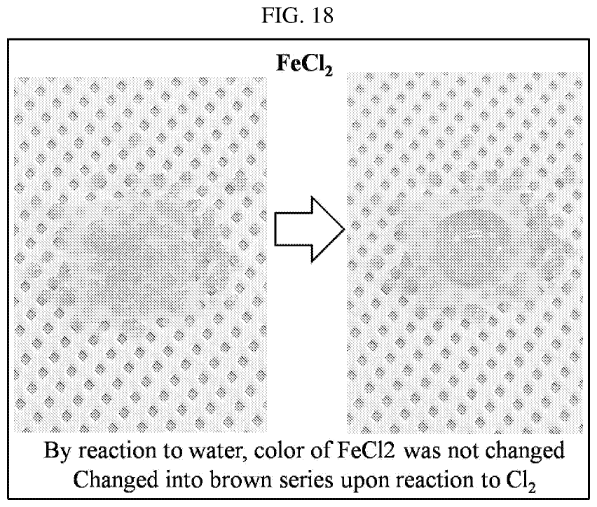

FIGS. 16 to 18 are images illustrating examples of a color change attributable to the influence of moisture in an embodiment of the present disclosure. The image in FIG. 16 illustrates an example in which the color of the colorimetric sensor using the DPD dye, which was changed into yellow due to the reaction with the Cl₂ gas, was changed into thin pink in a reaction with water. Furthermore, the image in FIG. 17 illustrates an example in which the color of the colorimetric sensor using copper acetate, which was changed into yellow due to the reaction with the Cl₂ gas, was changed into colorlessness in a reaction with water. Furthermore, the image in FIG. 18 illustrates that the colorimetric sensor using FeCl₂, which was changed into brown series due to the reaction with the Cl₂ gas, had not a change in color in a reaction with water. As described above, it can be seen that in the colorimetric sensor according to embodiments of the present disclosure, a color change attributable to the influence of moisture and a color change attributable to the reaction with the Cl₂ gas are quite different from each other.

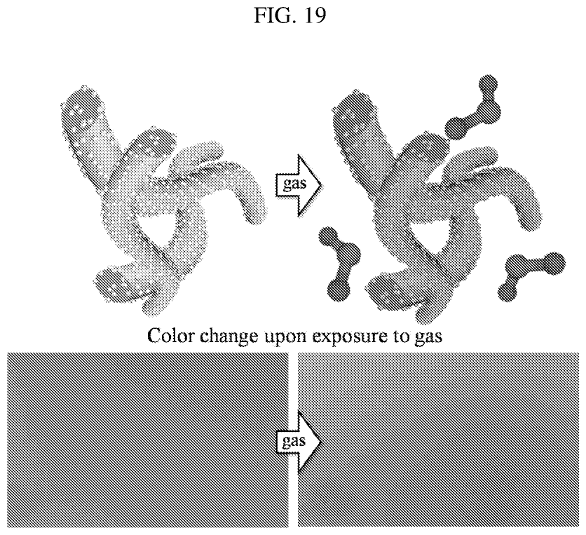

FIG. 19 is a diagram illustrating an example of a color change process of the submicron fiber membrane by a combination with a harmful gas in an embodiment of the present disclosure. FIG. 19 illustrates an example in which a three-dimensional submicron fiber membrane to which a dye having a color changed upon reaction with a harmful gas was bound is changed.

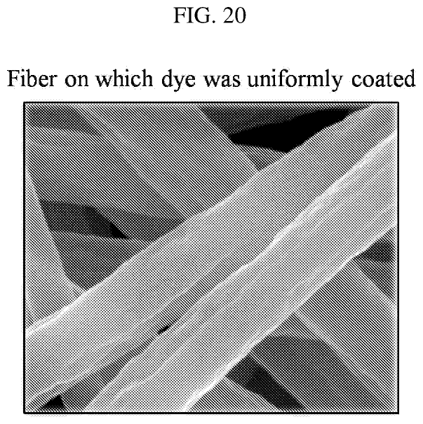

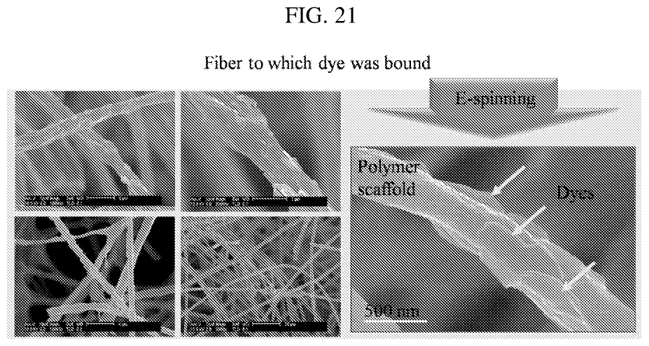

FIG. 20 is an image illustrating an example of a submicron fiber membrane on which a dye was uniformly coated in an embodiment of the present disclosure. FIG. 20 shows images illustrating an example of a submicron fiber membrane to which a dye was bound in an embodiment of the present disclosure. The image in FIG. 20 illustrates an example in which the dye was coated on a surface of a fiber having a submicron diameter in a uniform film form. The images in FIG. 21 illustrate an example in which the dye was bound to the inside and surface of a fiber having a submicron diameter in a particle form.

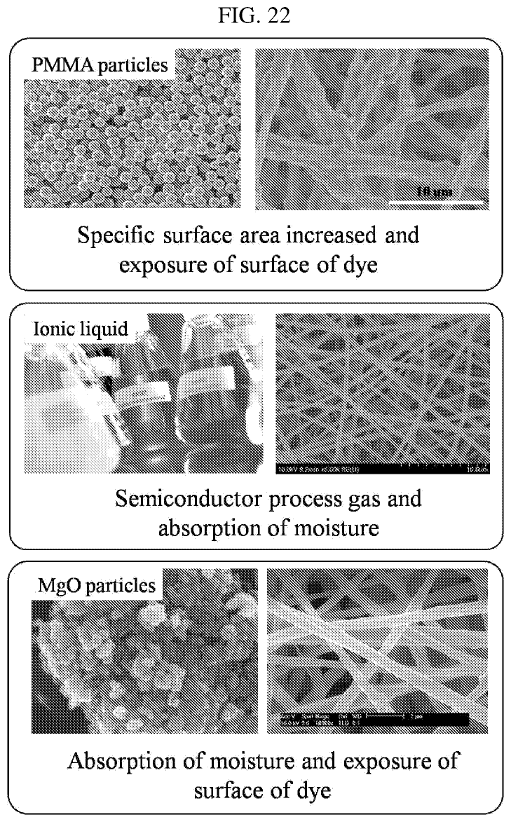

FIG. 22 is a diagram illustrating an example in which a colorimetric accelerator was used in an embodiment of the present disclosure. FIG. 22 illustrates that performance of a color change of the colorimetric sensor can be improved by using polymer beads such as polymethyl methacrylate (PMMA) particles, an ionic liquid, or ceramic particles such as MgO particles, as the colorimetric accelerator. For example, the PMMA particles that have been partially dissolved enable a colorimetric fiber membrane having a rough surface and a high specific surface area to be manufactured, so that the specific surface area can be increased and a degree of the exposure of a surface of a dye can be increased. Hydrophilic particles, such as the ionic liquid, can increase the adsorption of a gas or moisture. MgO particles have been known as a material that well absorbs moisture or carbon dioxide, and can help the adsorption of a gas or moisture and also increase a degree of the exposure of a surface of a dye. Performance of the colorimetric sensor may be different depending on a moisture an environment in addition to a shape of a fiber. Reactivity of the colorimetric sensor may be increased in an environment including a lot of moisture. Accordingly, if a material, such as MgO, is combined with a fiber as an additive, a degree of a color change or a colorimetric time may be increased because the fiber material itself can absorb moisture even in the same amount of gas and the same moisture environment.

The embodiments of the present disclosure can provide the colorimetric sensor capable of detecting the leakage of a harmful gas in a product manufacturing process, such as a semiconductor process or a display process, and the method of manufacturing the colorimetric sensor.

### MODE FOR DISCLOSURE

The above description is merely a description of the technical spirit of the present disclosure, and those skilled in the art may change and modify the present disclosure in various ways without departing from the essential characteristic of the present disclosure. Accordingly, the embodiments described in the present disclosure should not be construed as limiting the technical spirit of the present disclosure, but should be construed as describing the technical spirit of the present disclosure, and are not restricted by such embodiments. The range of protection of the present disclosure should be construed based on the following claims, and all of technical spirits within an equivalent range of the present disclosure should be construed as being included in the scope of rights of the present disclosure.

1. A colorimetric sensor comprising:
a colorimetric composition in which a dye comprising a metal component having a unique color changed upon reaction with a harmful gas has been bound to submicron fibers in a fine particle form; and
an adhesive band having a mesh structure to which the colorimetric composition has been bound.

2. The colorimetric sensor of claim 1, wherein the submicron fibers have a first fiber having a first thickness and a second fiber having a thickness relatively thicker than the first thickness intersected and formed, whereby a space for a spread and adsorption of a gas is formed between the first fibers by the second fiber.

3. The colorimetric sensor of claim 1, wherein at least some of the submicron fibers are aligned and formed in one direction in order to increase a density of the fibers.

4. The colorimetric sensor of claim 1, wherein:
the harmful gas comprises at least one of Cl₂, BCl₃, BF₃, HBr, SiH₄, NH₃, HCl steam, HF steam and TMAH steam,
the metal component comprises at least one of Cu, Co, Ni, Fe, Zn, Mn, and Pb as a form of an ion or salt, and
the dye further comprises at least one of an acetate group, a chlorine group, and a nitrate group.

5. The colorimetric sensor of claim 1, wherein the adhesive band having the mesh structure is bound to the colorimetric composition so that the adhesive band has a relatively wider area than the colorimetric composition in order to bind the colorimetric composition to a piping and processing facility for a product manufacturing process.

6. The colorimetric sensor of claim 1, wherein a colorimetric accelerator is further bound to the submicron fibers.

7. The colorimetric sensor of claim 6, wherein the colorimetric accelerator comprises at least one of particulates, polymeric beads, ceramic particles, and ionic liquids that provide surface texturing.

8. A method of manufacturing a colorimetric sensor, comprising steps of:
manufacturing a colorimetric composition comprising submicron fibers to which a dye comprising a metal component having a unique color changed upon reaction with a harmful gas has been bound; and
binding the colorimetric composition to an adhesive band having a mesh structure.

9. The method of claim 8, wherein the step of manufacturing the colorimetric composition comprises steps of:
manufacturing an electrospinning solution comprising a dye, polymers, and a solvent; and
manufacturing a submicron fiber membrane to which the dye has been bound in a fine particle form by electrospinning the electrospinning solution.

10. The method of claim 8, wherein the step of manufacturing the colorimetric composition comprises steps of:
manufacturing an electrospinning solution comprising polymers, and a solvent;
manufacturing a submicron fiber membrane by electrospinning the electrospinning solution; and
coating the dye on a fiber comprising the submicron fiber membrane.
